(19) United States
(12) Patent Application Publication
(10) Pub. No.: US 2010/0004587 A1
(43) Pub. Date: Jan. 7, 2010

(54) Nose, mouth and ear hydrogen peroxide delivery device

(76) Inventors: Gil G. Moreno, Tampa, FL (US); Gil Moreno, Tampa, FL (US)

Correspondence Address: Gil G. Moreno, 4106 Dellbrook Dr., Tampa, FL, 33624-1838, US

(21) Appl. No.: 12/215,883

(22) Filed: Jul. 1, 2008

(51) Int. Cl. A61M 37/00

(52) U.S. Cl. 604/25; 604/26

(57) ABSTRACT

This relates to a method to generate a mixture of Hydrogen Peroxide gas and a Carrier gas by passing dry Carrier gas through Hydrogen Peroxide.

### BACKGROUND OF THE INVENTION

Hydrogen Peroxide liquid has been used for long time as an effective means to destroy undesirable microorganisms on humans and living creatures. The nasal and vocal cavities are a paradise for said microorganisms.

Hydrogen Peroxide is a liquid at normal temperature and barometric pressure. Its boiling point is 150.2 grades centigrade, and is miscible with water. The present invention delivers hydrogen peroxide gas to nose, mouth and ears. As a gas the hydrogen peroxide diffuses and is capable of traveling to the most difficult locations.

The applicants set up tests to determine the efficiency of the present invention to deliver hydrogen peroxide gas to nose, mouth and ears. A carrier gas was passed thru a cell containing a solution of 3 percent Hydrogen Peroxide available in the drug stores. The cell output was far from the solution surface to allow only a mixture of carrier gas and hydrogen peroxide gas to be delivered to a solution of Potassium Permanganate, which normally is pink, turning it yellow that indicated the carrier gas contained Hydrogen Peroxide gas. The tests were performed at 90 and 70 percent relative humidity and the carrier gas was air but other carrier gases may be used.

During a hard breath-hold, the epiglottis closes the access to the lungs. Then the nose, mouth and ears can be irrigated with Hydrogen Peroxide rich gas. After irrigation the air in the lungs is expelled thru the nose and mouth to remove the Hydrogen Peroxide rich gas. Some Hydrogen Peroxide remains trapped in the mucus for a short period extending the germ killer action.

At present there is not a simple and practical device to irrigate the nose, mouth and ears with Hydrogen Peroxide rich gas.

U.S. Pat. No. 7,347,201 is complicated and would require extensive modifications to accomplish delivery of Hydrogen Peroxide gas.

U.S. Pat. No. 7,244,354 is extremely complicated and uses ozone dissolved in water or solution.

The present invention provides an economical and easy way to destroy the majority of harmful microorganisms infesting the nose, mouth and ears.

### SUMMARY OF THE INVENTION

The principal object of the present invention is to provide a device to irrigate the nose, mouth and ears with Hydrogen Peroxide rich gas that is simple, is portable and requires minimum mental effort to operate.

It is also an object of the present invention to provide a device, which is of inexpensive construction.

Another object of the present invention is to provide a device that is attractive and pleasant to use.

A further object is to provide a device that will contribute to a healthy life.

For accomplishing these objects a Hydrogen Peroxide rich gas is pumped into the nose, mouse or ears using a nozzle.

### BRIEF DESCRIPTION OF THE DRAWINGS

FIG. 1 shows the most relevant components of the device to irrigate the nose, mouth and ears with Hydrogen Peroxide rich gas.

FIG. 2 is a cross sectional view of the Hydrogen Peroxide rich gas delivery nozzle with a cylindrical cavity to accept the feeding hose, and protective cap.

FIG. 3 is a cross-sectional view of the Hydrogen Peroxide rich gas delivery nozzle with a cylindrical extension to fit inside the feeding hose, and protective cap.

### DESCRIPTION OF THE PREFERRED EMBODIMENTS

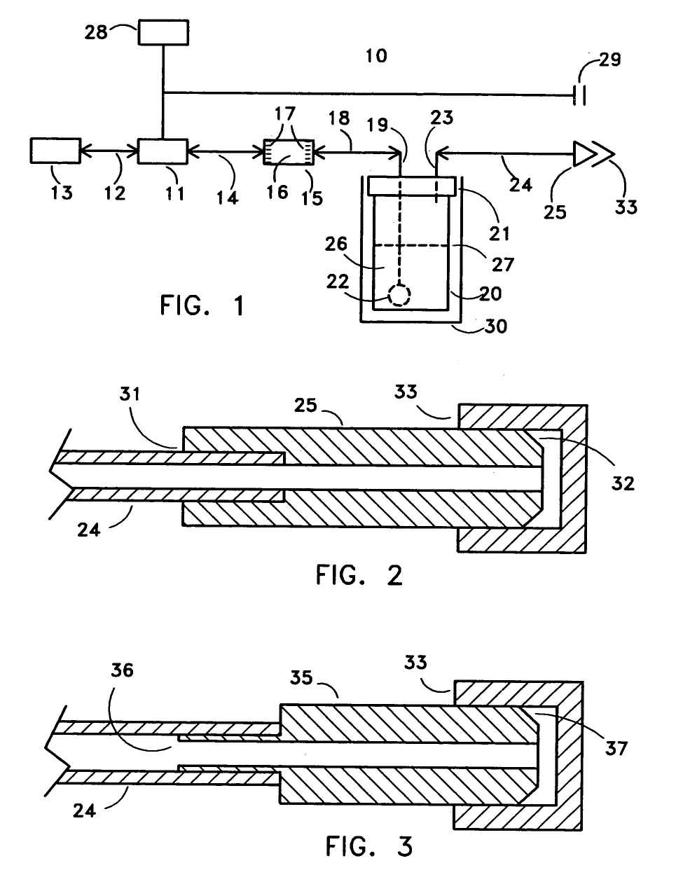

Referring to FIG. 1 the device generally designated 10 comprises a gas pump 11 connected with hose 12 to a gas filter 13. Said gas pump 11 is also connected with hose 14 to gas dryer cell 15 containing silica gel desiccant or equivalent 16. Said 15 has at entrance and exit wire mesh 17 to prevent escape of 16. Said 15 is connected with hose 18 to a first tube 19 of bubbling cell 20 made of translucent material. Said 19 penetrates lid 21 of 20 and has at the end an air stone 22 close to the bottom of said 20. One end of a second tube 23 is just long enough to penetrate said lid 21. The other end of 23 is connected to one end of hose 24, and the other end of 24 is connected to delivery nozzle 25 or 35. Cap 33 fits tight 25 and 35 to prevent access of dust and insects during storage. Said 20 is filled with solution 26 of hydrogen peroxide up to indicating mark 27 to allow only gas to exit thru said 24. Electric power 28 from batteries or utility service feeds said 11 via momentary normally open push button switch 29. Light shield 30 covers said 20 and can be removed during refilling to make visible said 27.

Referring to FIG. 2 nozzle 25 is of sufficient length and diameter to be held by fingers. One end of said 25 has a cylindrical cavity 31 to accept tight hose 24. The other end 32 of said 25 is conical to facilitate alignment, of diameter large enough to prevent penetration in human nostril and ears. A cap 33 fits tight to 25 to prevent access of dust and insects during storage.

Referring to FIG. 3 nozzle 35 is of sufficient length and diameter to be held by fingers. One end of said 35 has a cylindrical extension 36 to penetrate tight hose 24. The other end 37 of said 35 is conical to facilitate alignment, of diameter large enough to prevent penetration in human nostril and ears. A cap 33 fits tight to 35 to prevent access of dust and insects during storage.

Although the invention has been disclosed in its prefer forms with a certain degree of particularity, it is understood that the invention of the preferred forms has been made by way of examples, that numerous changes in the detail of construction and the combination and arrangement of parts may be resorted to without departing from the spirit and scope of the invention.

1-7. (canceled)

8. A method to generate a mixture of Hydrogen Peroxide gas and a Carrier gas consisting in passing a dry Carrier gas through Hydrogen Peroxide solution.
